(19) United States
(12) Patent Application Publication
(10) Pub. No.: US 2006/0217347 A1
(43) Pub. Date: Sep. 28, 2006

(54) Topical formulations of borinic acid antibiotics and their methods of use

(76) Inventors: David Perry, Palo Alto, CA (US); Kirk R. Maples, San Jose, CA (US); Meidong Yang, Petaluma, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(21) Appl. No.: 11/389,605

(22) Filed: Mar. 24, 2006

Related U.S. Application Data
(60) Provisional application No. 60/665,178, filed on Mar. 24, 2005.

(51) Int. Cl. A61K 31/69

(52) U.S. Cl. 514/64

(57) ABSTRACT

Disclosed are topical formulations, for example, creams, gels, or lotions, of borinic acid antibiotic compounds active against acne vulgaris or secondarily infected skin conditions.

### FIELD OF THE INVENTION

This invention relates to topical pharmaceutical formulations. More specifically, the present invention is directed to creams, gels, or lotion formulations of borinic acid antibiotic compounds having activity against acne vulgaris or secondarily infected skin conditions. Thus, the present invention relates to the fields of pharmacy, dermatology, and medicinal chemistry.

### REFERENCES

The following publications are cited throughout this application using the first author's last name and the publication year:
1. Eady, A. E., Cove, J. H., Layton, A. M.; “Is antibiotic resistance in cutaneous propionibacteria clinical relevant?: implications of resistance for acne patients and prescribers;” American J. Clin. Derm., 4, pages 813-831, (2003).
2. Harrison, J. E., Watkinson, A. C., Green, D. M., Hadgraft, J., Brian, K.; “The relative effect of Azone and Transcutol on permeant diffusivity and solubility in human stratum corneum;” Pharm. Res., 13, pages 542-546, (1996).
3. Osborne, D. W., Henke, J. J.; “Skin penetration enhancers cited in the technical literature;” Pharm. Tech., 21, pages 58-66, (1997).
4. Purdon, C. H., Azzi, C. G., Zhang, J., Smith, E. W., Maibach, H. I.; “Penetration enhancement of transdermal delivery—current permutations and limitations;” Crit. Rev. Therap. Drug Carrier Sys., 21, pages 97-132, (2004).
5. Ritschel, W. A., Panchagnula, R., Stemmer, K., Ashraf, M.; “Development of an intracutaneous depot for drugs. Binding, drug accumulation and retention studies, and mechanism of depot;” Skin Pharmacol., 4, pages 235-245, (1991).
6. Rojas, J., Falson, F., Couarraze, G., Francis, A., Puisieux, F.; “Optimization of binary and ternary solvent systems in the percutaneous absorption of morphine base;” STP Pharma Sciences, 1, pages 70-75, (1991).
7. Watkinson, A. C., PhD Thesis, University of Wales, 1991.
8. Watkinson, A. C., Hadgraft, J., Bye, A.; “Aspects of the transdermal delivery of prostaglandins;” Int. J. Pharm., 74, pages 229-236, (1991).
9. Williams, A. C., Barry, B. W.; “Penetration enhancers;” Adv. Drug Deliv. Rev., 56, pages 603-618, (2004).

Each of the above publications and applications is incorporated herein by reference in its entirety and for all purposes.

### STATE OF THE ART

Acne vulgaris, generally called “acne”, is the most common skin disease of adolescence and early adulthood. Estimates indicate that approximately 85% of all adolescents experience some degree of acne, and recent studies have shown that approximately 17 million Americans are affected by this condition. Although generally considered a condition of adolescence, acne also affects 8% of Americans between 24- and 34 years of age and 3% of those 35- to 44 years of age. Studies have also shown that acne vulgaris can directly and significantly affect the patient's quality of life, including causing such psychological effects as anxiety, depression and withdrawal from society.

Patients suffering from acne have been treated with antibiotic agents for more than 30 years. These agents are believed to exert their therapeutic effects by reducing the population of Propionibacterium acnes (P. acnes) and its associated mediators of inflammation as well as by having direct anti-inflammatory actions. The main concern with the use of antibiotics for the treatment of acne is the inevitable emergence of resistant organisms. Antibiotic resistance, once thought of as uncommon, is now recognized as a clinically significant problem: a systematic review of published studies has shown that the overall incidence of antibiotic-resistant P. acnes has increased from 20% in 1978 to 62% in 1996. Eady et al. (2003), have shown that resistant strains of P. acnes are associated with treatment failure. In addition, since the skin is a potent site for the exchange of antimicrobial resistance, the danger of spreading antibiotic resistance to other species, such as staphylococci, is a very real cause for concern.

Staphylococcus aureus (S. aureus) is a frequent cause of secondary infection in skin conditions, such as atopic dermatitis. Patients with atopic dermatitis are colonized with S. aureus, and the organism has been isolated from infected eczema, from chronic lesions, and from clinically normal skin in patients with atopic dermatitis. S. aureus may also influence disease activity in the absence of overt clinical infection and exacerbate eczema by several mechanisms. Protein A, contained in the S. aureus cell wall, causes a biphasic reaction resulting in an initial wheal and flare and a late indurated response when injected intradermally. Circulating antistaphylococcal IgE antibodies have been found in up to 30% of patients with atopic dermatitis, and these may cause mast cell degranulation via an IgE-mediated reaction. S. aureus isolated from patients with atopic dermatitis has the ability to produce superantigens. These cause inflammation by activating T-lymphocyte and macrophages, releasing cytokines, causing mast cell degranulation, and enhancing IgE-mediated reactions.

Untreated severe acne may result in permanent scarring. Topical medication is generally adequate in clearing comedonal acne, while inflammatory acne usually requires the addition of oral medication. Treatment may also require a combination of acne treatment products, depending on the specific condition, genetic nature and severity of the disease. It is very important for the acne patient to consult with a physician if acne is severe. Effective treatment of acne vulgaris can prevent emotional and physical scarring.

Thus, there is a need for new topical formulations for treating acne, superficial skin infections, pathogen colonization and inflammatory skin conditions. Such treatments preferably do not lead to pathogen resistance with repeated use.

### SUMMARY OF THE INVENTION

This invention provides novel formulations of borinic acid antibiotic compounds that are active against acne vulgaris and/or secondarily infected skin conditions. More specifically, the invention provides topical pharmaceutical formulations comprising a pharmaceutically acceptable topical carrier and a compound of Formula 1 or its pharmaceutically acceptable salt form: 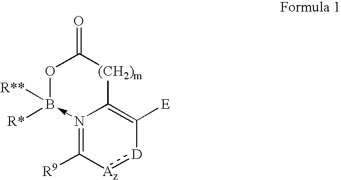
R* and R** are members independently selected from substituted or unsubstituted aralkyl, substituted or unsubstituted aryl, substituted or unsubstituted cycloalkyl, and substituted or unsubstituted heterocycle. The variable z is 0 or 1, with the proviso, that if z is 1, then A is a member selected from CR¹⁰ and N, and D is a member selected from N and CR¹², and with the further proviso that if z is 0, then D is a member selected from O, S and NR^{12a}. E is a member selected from hydrogen, hydroxy, alkoxy, (cycloalkyl)oxy, (cycloheteroalkyl)oxy, carboxy, or alkyloxycarbonyl. The variable m is 0 or 1. R¹² is a member selected from hydrogen, hydroxyalkyl, aminoalkyl, alkylaminoalkyl, dialkylaminoalkyl, carboxy, alkyloxycarbonyl, amido, hydroxy, alkoxy, aryloxy, thio, alkylthio, arylthio, alkylsulfonyl, dialkylaminosulfonyl, alkylaminosulfonyl, aminosulfonyl, sulfo, cyano, halo, nitro, amino, dialkylamino, alkylamino, arylamino, diarylamino, aralkylamino, and diaralkylamino. In addition, the alkyl or aryl portion of any moiety recited for R¹² is optionally substituted. R^{12a} is a member selected from hydrogen, substituted or unsubstituted alkyl, substituted or unsubstituted heteroalkyl, substituted or unsubstituted aralkyl, substituted or unsubstituted aryl, substituted or unsubstituted cycloalkyl, and substituted or unsubstituted heterocycle; R⁹ and R¹⁰ are members independently selected from hydrogen, alkyl, cycloalkyl, hydroxyalkyl, aminoalkyl, alkylaminoalkyl, dialkylaminoalkyl, halo, carbonyl, hydroxyimino, carboxy, alkyloxycarbonyl, alkylthio, alkylsulfonyl, arylthio, dialkylaminosulfonyl, alkylaminosulfonyl, aminosulfonyl, amino, alkoxy, nitro, sulfo, and hydroxy. The alkyl or aryl portion of any moiety recited for R⁹ or R¹⁰ is optionally substituted.

In some embodiments, E is a member selected from hydrogen, hydroxy, or (cycloheteroalkyl)oxy such as 2-morpholinoethoxy. In other embodiments, R¹² is (CH₂)ₖOH (where k=1, 2 or 3), CH₂NH₂, CH₂NH-alkyl, CH₂N(alkyl)₂, CO₂H, CO₂alkyl, CONH₂, OH, alkoxy, aryloxy, SH, S-alkyl, S-aryl, SO₂alkyl, SO₂N(alkyl)₂, SO₂NHalkyl, SO₂NH₂, SO₃H, SCF₃, CN, halogen, CF₃, NO₂, NH₂, 2°-amino, 3°-amino, NH₂SO₂ and CONH₂. In still other embodiments, R⁹ and R¹⁰ are hydrogen, alkyl, cycloalkyl, (CH₂)ₙOH (n=1 to 3), CH₂NH₂, CH₂NHalkyl, CH₂N(alkyl)₂, halogen, CHO, CH═NOH, CO₂H, CO₂-alkyl, S-alkyl, SO₂-alkyl, S-aryl, SO₂N(alkyl)₂, SO₂NHalkyl, SO₂NH₂, NH₂, alkoxy, CF₃, SCF₃, NO₂, SO₃H and OH;

In Formula 1 the illustrated dative bond (arrow) may or may not be present, i.e., the present invention includes those compounds in which coordination between the boron atom and the nitrogen of the picolinate ring is present and those compounds where such coordination is missing. The present invention also includes those compounds of Formula 1 in which a dative bond is formed between the boron and another atom of the molecule. In addition, those of skill in the art, e.g., organic and medicinal chemistry, will appreciate that the large difference in atomic radius between carbon and boron can allow for the formation of solvent coordination complexes in which a solvent molecule, such as water, can be inserted between the boron atom and the nitrogen atom of the picolinate ring. The present invention includes such adducts of the compounds of Formula 1.

In one embodiment of the invention in which z is 1, the compound of Formula 1 has a structure according to the following formula: 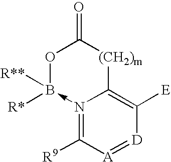
wherein D is a member selected from N and CR¹².

In another embodiment of the invention, in which z is 0, the compound of Formula 1 has a structure according to the following formula: 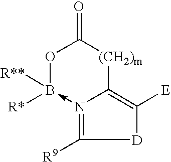
wherein D is a member selected from O, S and NR^{12a}.

In one embodiment of the invention, R* and R** are the same. In a more specific embodiment, R* and R** are substituted or unsubstituted aryl. In a still more specific embodiment, R* and R** are substituted or unsubstituted phenyl, wherein said substituted or unsubstituted phenyl has the structure: 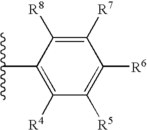
and further wherein each of R⁴-R⁸ is a member independently selected from hydrogen, alkyl, cycloalkyl, aryl, substituted aryl, aralkyl, substituted aralkyl, hydroxyalkyl, aminoalkyl, alkylaminoalkyl, dialkylaminoalkyl, carboxy, alkylcarbonyl, aminocarbonyl, alkylaminocarbonyl, dialkylaminocarbonyl, hydroxy, alkoxy, aryloxy, thio, alkylthio, arylthio, alkylsulfonyl, diaminosulfonyl, alkylaminosulfonyl, aminosulfonyl, sulfo, cyano, halo, nitro, amino, 2′-amino, 3′-amino, aminosulfonyl, aminoalkyloxy, (alkylamino)alkyloxy, (dialkylamino)alkyloxy, and cycloheteroalkyl. Each alkyl or aryl portion of each moiety recited for R⁴-R⁸ is optionally substituted. In more specific embodiments of the invention in which R* and R** are both optionally substituted phenyl as just described, each of R⁴-R⁸ is a member independently selected from the group consisting of: hydrogen, alkyl, cycloalkyl, aryl, substituted aryl, aralkyl, substituted aralkyl, (CH₂)ₖOH (where k=1, 2 or 3), CH₂NH₂, CH₂NH-alkyl, CH₂N(alkyl)₂, CO₂H, CO₂alkyl, CONH₂, CONHalkyl, CON(alkyl)₂, OH, alkoxy, aryloxy, SH, S-alkyl, S-aryl, SO₂alkyl, SO₂N(alkyl)₂, SO₂NHalkyl, SO₂NH₂, SO₃H, SCF₃, CN, halogen, CF₃, NO₂, NH₂, 2°-amino, 3°-amino, NH₂SO₂, OCH₂CH₂NH₂, OCH₂CH₂NHalkyl, OCH₂CH₂N(alkyl)₂, oxazolidin-2-yl, and alkyl substituted oxazolidin-2-yl.

In one embodiment of the invention in which R* and R** are both optionally substituted phenyl as described, R⁹ is H, z is 1, A is CH, D is CH, E is OH, and m is 0. In a more specific embodiment of the foregoing, R* and R** are both 3-chloro-4-methylphenyl. In another specific embodiment, R* and R** are both 2-methyl-4-chlorophenyl. Particularly useful compounds include 3-hydroxypyridine-2-carbonyloxy-bis(3-chloro-4-methylphenyl)-borane and 3-hydroxypyridine-2-carbonyloxy-bis(2-methyl-4-chlorophenyl)-borane, and their pharmaceutically acceptable salts.

In one embodiment, the pharmaceutically acceptable topical carrier comprises at least one solvent in which a compound of Formula I is soluble. Preferably, the compound of Formula I has a solubility of at least about 10% wt/wt in one solvent. This solvent is also preferably miscible with water. A preferred water miscible solvent for the present invention is diethylene glycol monoethyl ether. The solvent of the present invention may also be a mixture of solvents, for example water and diethylene glycol monoethyl ether, or alcohol and water. According to one useful embodiment of the invention, the solvent mixture contains water in an amount that allows the amount of the compound dissolved in the solvent mixture to be sufficient to create a topical pharmaceutical formulation that includes at least about 1% of the compound (wt/wt).

In one embodiment, the topical pharmaceutical formulation comprises a compound of Formula I, at least one solvent, and at least one emulsifier.

In another embodiment, the topical pharmaceutical formulation comprises at least one solvent, at least one emollient, at least one antioxidant, at least one emulsifier, at least one preservative, at least one chelating agent, at least one viscosity increasing agent, at least one neutralizing agent in combination with a compound of Formula I. Preferably the topical pharmaceutical formulation comprises a compound of Formula I, cetyl alcohol, isopropyl myristate, stearyl alcohol, butylated hydroxytoluene, polyoxyethylene (2) stearyl ether (Brij 72), polyoxyethylene (21) stearyl ether (Brij 721), methylparaben, propylparaben, EDTA, diethylene glycol monoethyl ether, CARBOPOL ULTREZ 10, 25% trolamine solution, and water.

In another embodiment, the topical pharmaceutical formulation comprises a keratinization-modifying agent.

This invention also provides methods for treating a patient with a dermatological condition in which a therapeutically effective amount of the topical pharmaceutical formulation as described herein is administered topically to such a patient. In particular examples of such methods, the dermatological condition may be an inflammatory condition, acne, pruritis, or a secondarily infected skin condition (including impetigo and atopic dermatitis).

In another embodiment, the topical pharmaceutical formulation comprises at least one solvent, at least one emollient, at least one antioxidant, at least one emulsifier, at least one preservative, at least one chelating agent, at least one viscosity increasing agent, at least one neutralizing agent, and water. In an exemplary embodiment, the emollient is a mixture of cetyl alcohol, isopropyl myristate and stearyl alcohol. In an exemplary embodiment, the antioxidant is butylated hydroxytoluene. In an exemplary embodiment, the emulsifier is a combination of polyoxyethylene (2) stearyl ether (Brij 72) and polyoxyethylene (21) stearyl ether (Brij 721). In an exemplary embodiment, the preservative is a combination of methylparaben and propylparaben. In an exemplary embodiment, the viscosity increasing agent is diethylene glycol monoethyl ether and/or CARBOPOL ULTREZ 10. In an exemplary embodiment, the chelating agent is EDTA. In another exemplary embodiment, trolamine is in a 25% trolamine solution. In an exemplary embodiment, this topical pharmaceutical formulation can be used to treat pruritis, or itching.

### DETAILED DESCRIPTION OF THE INVENTION

The invention is directed to novel formulations of borinic acid antibiotic compounds that are active against acne vulgaris and/or secondarily infected atopic dermatitis. However, prior to describing this invention in detail, the following terms will first be defined:

Definitions

By “alkyl”, “lower alkyl”, and “C₁-C₆ alkyl” in the present invention is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

By “alkoxy”, “lower alkoxy”, and “C₁-C₆ alkoxy” in the present invention is meant straight or branched chain alkoxy groups having 1-6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

By the term “halogen” in the present invention is meant fluorine, bromine, chlorine, and iodine.

By “cycloalkyl”, e.g., C₃-C₇ cycloalkyl, in the present invention is meant cycloalkyl groups having 3-7 atoms such as, for example cyclopropyl, cyclobutyl, cyclopentyl, cyclohexyl, and cycloheptyl. In C₃-C₇ cycloalkyl groups, preferably in C₅-C₇ cycloalkyl groups, one or two of the carbon atoms forming the ring can be replaced with a hetero atom, such as sulfur, oxygen or nitrogen. Examples of such groups are pipendinyl, piperazinyl, morpholinyl, pyrrolidinyl, imidazolidinyl, oxazolidinyl, perhydroazepinyl, perhydrooxazapinyl, oxepanyl, and perhydrooxepanyl. C₃ and C₄ cycloalkyl groups having a member replaced by nitrogen or oxygen include aziridinyl, azetidinyl, oxetanyl, and oxiranyl.

“Cycloheteroalkyl” is defined as “cycloalkyl” above, but wherein at least one atom of the ring is a heteroatom, such as nitrogen, sulfur, or oxygen. Examples of cycloheteroalkyl groups include, but are not limited to: furanyl, piperazyl, thiophenyl, pyranyl and the like.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), which is optionally mono-, di-, or trisubstituted with, e.g., halogen, lower alkyl, lower alkoxy, lower alkylthio, trifluoromethyl, lower acyloxy, aryl, heteroaryl, and hydroxy. Preferred aryl groups include phenyl and naphthyl, each of which is optionally substituted as defined herein.

By “heteroaryl” is meant one or more aromatic ring systems of 5-, 6-, or 7-membered rings containing at least one and up to four heteroatoms selected from nitrogen, oxygen, or sulfur. Such heteroaryl groups include, for example, thienyl, furanyl, thiazolyl, imidazolyl, oxazolyl, isoxazolyl, pyridyl, pyrimidinyl, quinolinyl, isoquinolinyl, napthyridinyl, benzimidazolyl, and benzoxazolyl. Preferred heteroaryls are thiazolyl, pyrimidinyl, preferably pyrimidin-2-yl, and pyridyl. Other preferred heteroaryl groups include 1-imidazolyl, 2-thienyl, 1-(or 2-)quinolinyl, 1-(or 2-)isoquinolinyl, 1-(or 2-)tetrahydroisoquinolinyl, 2-(or 3-)furanyl and 2-tetrahydrofuranyl.

Heterocyclo” refers generically both to cycloheteroalkyl and heteroaryl as defined herein.

The terms “drug,” “active agent,” “active ingredient” or “pharmaceutical agent” refer to any chemical material, compound or composition suitable for topical or transdermal administration which provides a desired biological, pharmacological or nutritional effect. These terms are also meant to include mixtures of more than one active agent.

The term “aralkyl” refers to aryl-alkyl-, where the aryl group is bound to the core structure through an alkyl group.

The term “aryloxy” refers to an aryl-O— group.

The term “amino” refers to the —NH₂ group. The term “secondary amino” or “2°-amino” means —NHR, where R is selected from optionally substituted alkyl, heteroalkyl, cycloalkyl, heterocyclic, aryl, heteroaryl, and the like. The term “tertiary amino” or “3°-amino” means —NRR′, where each R and R′ is selected from optionally substituted alkyl, heteroalkyl, cycloalkyl, heterocyclic, aryl, heteroaryl, and the like.

The term “optionally substituted” refers to groups that can either be substituted or unsubstituted. A substituted group preferably has from 1 to 3 substituents selected from nitro, cyano, halogen, optionally substituted alkyl, heteroalkyl, cycloalkyl, heterocyclic, aryl, heteroaryl, amino, thiol, alkylthiol, alkoxy, acyl, acylamino, aminoacyl, carboxyl, carboylester and SO₂—R, where R is selected from optionally substituted alkyl, heteroalkyl, cycloalkyl, heterocyclic, aryl, and heteroaryl.

By “effective” amount of a drug, formulation, or permeant is meant a sufficient amount of an active agent to provide the desired local or systemic effect. A “Topically effective,” “Cosmetically effective,” “pharmaceutically effective,” or “therapeutically effective” amount refers to the amount of drug needed to effect the desired therapeutic result.

“Topically effective” refers to a material that, when applied to the skin, produces a desired pharmacological result either locally at the place of application or systemically as a result of transdermal passage of an active ingredient in the material.

“Cosmetically effective” refers to a material that, when applied to the skin, produces a desired cosmetic result locally at the place of application of an active ingredient in the material.

“Pharmaceutically acceptable salts” refers to pharmaceutically acceptable salts of a compound, which salts are derived from a variety of organic and inorganic counter ions well known in the art and include, by way of example only, hydrochloric, phosphoric, hydrobromic, sulfuric, sulfinic, formic, toluenesulfonic, methanesulfonic, hydroxyethanesulfonic, nitric, benzoic, citric, tartaric, maleic, fumaric hydroiodic, lactic, succinic, alkanoic such as acetic, HOOC—(CH₂)ₚ—CH₃ where p is 0-4, and the like. In addition, pharmaceutically compatible salts can be formed with many acids, including but not limited to non-toxic pharmaceutical base addition salts including salts of bases such as sodium, potassium, calcium, ammonium, and the like. Those skilled in the art will recognize a wide variety of non-toxic pharmaceutically acceptable addition salts.

The term “pharmaceutically acceptable carrier” or “pharmaceutically acceptable vehicle” refers to any formulation or carrier medium that provides the appropriate delivery of an effective amount of a active agent as defined herein, does not interfere with the effectiveness of the biological activity of the active agent, and that is sufficiently non-toxic to the host or patient. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Part 8 of Remington's Pharmaceutical Sciences, 17.sup.th edition, 1985, Mack Publishing Company, Easton, Pa., which is incorporated herein by reference.

“Pharmaceutically acceptable topical carrier” and equivalent terms refer to pharmaceutically acceptable carriers, as described herein above, suitable for topical application; An inactive liquid or cream vehicle capable of suspending or dissolving the active agent(s), and having the properties of being nontoxic and non-inflammatory when applied to the skin is an example of a pharmaceutically-acceptable topical carrier. This term is specifically intended to encompass carrier materials approved for use in topical cosmetics as well.

The term “pharmaceutically acceptable additive” refers to preservatives, antioxidants, fragrances, emulsifiers, dyes and excipients known or used in the field of drug formulation and that do not unduly interfere with the effectiveness of the biological activity of the active agent, and that is sufficiently non-toxic to the host or patient. Additives for topical formulations are well-known in the art, and may be added to the topical composition, as long as they are pharmaceutically acceptable and not deleterious to the epithelial cells or their function. Further, they should not cause deterioration in the stability of the composition. For example, inert fillers, anti-irritants, tackifiers, excipients, fragrances, opacifiers, antioxidants, gelling agents, stabilizers, surfactant, emollients, coloring agents, preservatives, buffering agents, other permeation enhancers, and other conventional components of topical or transdermal delivery formulations as are known in the art.

The terms “enhancement,” “penetration enhancement” or “permeation enhancement” relate to an increase in the permeability of the skin to a drug, so as to increase the rate at which the drug permeates through the skin. The enhanced permeation effected through the use of such enhancers can be observed, for example, by measuring the rate of diffusion of the drug through animal or human skin using a diffusion cell apparatus. A diffusion cell is described by Merritt et al. Diffusion Apparatus for Skin Penetration, J. of Controlled Release, 1 (1984) pp. 161-162. The term “permeation enhancer” or “penetration enhancer” intends an agent or a mixture of agents, which, alone or in combination, act to increase the permeability of the skin to a drug.

The term “excipients” is conventionally known to mean carriers, diluents and/or vehicles used in formulating drug compositions effective for the desired use.

The term “topical administration” refers to the application of a pharmaceutical agent to the external surface of the skin, such that the agent crosses the external surface of the skin and enters the underlying tissues. Topical administration includes application of the composition to intact skin, to broken, raw or open wound of skin. Topical administration of a pharmaceutical agent can result in a limited distribution of the agent to the skin and surrounding tissues or, when the agent is removed from the treatment area by the bloodstream, can result in systemic distribution of the agent.

The term “transdermal delivery” refers to the diffusion of an agent across the barrier of the skin resulting from topical administration or other application of a composition. The stratum corneum acts as a barrier and few pharmaceutical agents are able to penetrate intact skin. In contrast, the epidermis and dermis are permeable to many solutes and absorption of drugs therefore occurs more readily through skin that is abraded or otherwise stripped of the stratum corneum to expose the epidermis. Transdermal delivery includes injection or other delivery through any portion of the skin or mucous membrane and absorption or permeation through the remaining portion. Absorption through intact skin can be enhanced by placing the active agent in an appropriate pharmaceutically acceptable vehicle before application to the skin. Passive topical administration may consist of applying the active agent directly to the treatment site in combination with emollients or penetration enhancers. As used herein, transdermal delivery is intended to include delivery by permeation through or past the integument, i.e. skin, hair, or nails.

Active Agents

Active agents useful in the presently claimed topical formulations are compounds that are active against acne vulgaris and/or secondarily infected skin conditions. Examples of active agents useful in the presently claimed topical formulations are disclosed in U.S. patent application Ser. No. 10/867,465 filed on Jun. 16, 2004, which application is incorporated herein in its entirety. Preferably the active agents are the borinic acid complexes of Formula I described herein above.

The present invention is directed to topical pharmaceutical compositions. These topical pharmaceutical compositions can be manufactured in a manner that is itself known, e.g., by means of a conventional mixing, dissolving, granulating, dragee-making, levigating, emulsifying, encapsulating, entrapping or lyophilizing processes.

Pharmaceutical compositions for use in accordance with the present invention thus can be formulated in conventional manner using one or more physiologically acceptable carriers comprising excipients and auxiliaries that facilitate processing of the active compounds into preparations that can be used pharmaceutically. Proper formulation is dependent upon the desired product chosen.

Formulations

The compositions of the present invention comprises fluid or semi-solid vehicles that may include but are not limited to polymers, thickeners, buffers, neutralizers, chelating agents, preservatives, surfactants or emulsifiers, antioxidants, waxes or oils, emollients, sunscreens, and a solvent or mixed solvent system. The solvent or mixed solvent system is important to the formation because it is primarily responsible for dissolving the drug. The best solvent or mixed solvent systems are also capable of maintaining clinically relevant levels of the drug in solution despite the addition of a poor solvent to the formulation. The topical compositions useful in the subject invention can be made into a wide variety of product types. These include, but are not limited to, lotions, creams, gels, sticks, sprays, ointments, pastes, foams, mousses, and cleansers. These product types can comprise several types of carrier systems including, but not limited to particles, nano-particles, and liposomes. If desired, disintegrating agents can be added, such as the cross-linked polyvinyl pyrrolidone, agar or alginic acid or a salt thereof such as sodium alginate. Techniques for formulation and administration can be found in “Remington's Pharmaceutical Sciences.” Mack Publishing Co, Easton, Pa. The formulation can be selected to maximize delivery to a desired target site in the body.

Lotions, which are preparations that are to be applied to the skin surface without friction, are typically liquid or semi-liquid preparations in which finely divided solid, waxy, or liquid are dispersed. Lotions will typically contain suspending agents to produce better dispersions as well as compounds useful for localizing and holding the active agent in contact with the skin, e.g., methylcellulose, sodium carboxymethyl-cellulose, or the like.

Creams containing the active agent for delivery according to the present invention are viscous liquid or semisolid emulsions, either oil-in-water or water-in-oil. Cream bases are water-washable, and contain an oil phase, an emulsifier and an aqueous phase. The oil phase is generally comprised of petrolatum or a fatty alcohol, such as cetyl- or stearyl alcohol; the aqueous phase usually, although not necessarily, exceeds the oil phase in volume, and generally contains a humectant. The emulsifier in a cream formulation, as explained in Remington: The Science and Practice of Pharmacy, supra, is generally a nonionic, anionic, cationic or amphoteric surfactant.

Gel formulations can also be used in connection with the present invention. As will be appreciated by those working in the field of topical drug formulation, gels are semisolid. Single-phase gels contain organic macromolecules distributed substantially uniformly throughout the carrier liquid, which is typically aqueous, but also may be a solvent or solvent blend.

Ointments, which are semisolid preparations, are typically based on petrolatum or other petroleum derivatives. As will be appreciated by the ordinarily skilled artisan, the specific ointment base to be used is one that provides for optimum delivery for the active agent chosen for a given formulation, and, preferably, provides for other desired characteristics as well, e.g., emolliency or the like. As with other carriers or vehicles, an ointment base should be inert, stable, nonirritating and non-sensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be had to Remington: The Science and Practice of Pharmacy, supra, for further information.

Useful formulations of the invention also encompass sprays. Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the drug or active agent can be dissolved. Upon delivery to the skin, the carrier evaporates, leaving concentrated active agent at the site of administration.

The topical pharmaceutical compositions may also comprise suitable solid or gel phase carriers. Examples of such carriers include but are not limited to calcium carbonate, calcium phosphate, various sugars, starches, cellulose derivatives, gelatin, and polymers such as polyethylene glycols.

The topical pharmaceutical compositions may also comprise a suitable emulsifier which refers to an agent that enhances or facilitates mixing and suspending oil-in-water or water-in-oil. The emulsifying agent used herein may consist of a single emulsifying agent or may be a nonionic, anionic, cationic or amphoteric surfactant or blend of two or more such surfactants; preferred for use herein are nonionic or anionic emulsifiers. Such surface-active agents are described in “McCutcheon's Detergent and Emulsifiers,” North American Edition, 1980 Annual published by the McCutcheon Division, MC Publishing Company, 175 Rock Road, Glen Rock, N.J. 07452, USA.

Preferred for use herein are high molecular weight alcohols such as cetearyl alcohol, cetyl alcohol, stearyl alcohol, emulsifying wax, glyceryl monostearate. Other examples are ethylene glycol distearate, sorbitan tristearate, propylene glycol monostearate, sorbitan monooleate, sorbitan monostearate (SPAN 60), diethylene glycol monolaurate, sorbitan monopalmitate, sucrose dioleate, sucrose stearate (CRODESTA F-160), polyoxyethylene lauryl ether (BRIJ 30), polyoxyethylene (2) stearyl ether (BRIJ 72), polyoxyethylene (21) stearyl ether (BRIJ 721), polyoxyethylene monostearate (Myrj 45), polyoxyethylene sorbitan monostearate (TWEEN 60), polyoxyethylene sorbitan monooleate (TWEEN 80), polyoxyethylene sorbitan monolaurate (TWEEN 20) and sodium oleate. Cholesterol and cholesterol derivatives may also be employed in externally used emulsions and promote w/o emulsions.

Especially suitable nonionic emulsifying agents are those with hydrophile-lipophile balances (HLB) of about 3 to 6 for w/o system and 8 to 18 for o/w system as determined by the method described by Paul L. Lindner in “Emulsions and Emulsion”, edited by Kenneth Lissant, published by Dekker, New York, N.Y., 1974, pages 188-190. More preferred for use herein are one or more nonionic surfactants that produce a system having HLB of about 8 to about 18.

Examples of such nonionic emulsifiers include but are not limited to “BRIJ 72”, the trade name for a polyoxyethylene (2) stearyl ether having an HLB of 4.9; “BRIJ 721”, the trade name for a polyoxyethylene (21) stearyl ether having an HLB of 15.5, “Brij 30”, the trade name for polyoxyethylene lauryl ether having an HLB of 9.7; “Polawax”, the trade name for emulsifying wax having an HLB of 8.0; “Span 60”, the trade name for sorbitan monostearate having an HLB of 4.7; “Crodesta F-160”, the trade name for sucrose stearate” having an HLB of 14.5. All of these materials are available from Ruger Chemicals Inc.; Croda; ICI Americas, Inc.; Spectrum Chemicals; and BASF. When the topical formulations of the present invention contain at least one emulsifying agent, each emulsifying agent is present in amount from about 0.5 to about 2.5 wt %, preferably 0.5 to 2.0%, more preferably 1.0% or 1.8%. Preferably the emulsifying agent comprises a mixture of steareth 21 (at about 1.8%) and steareth 2 (at about 1.0%).

The topical pharmaceutical compositions may also comprise suitable emollients. Emollients are materials used for the prevention or relief of dryness, as well as for the protection of the skin. Useful emollients include, but are not limited to, cetyl alcohol, isopropyl myristate, stearyl alcohol, and the like. A wide variety of suitable emollients are known and can be used herein. See e.g., Sagarin, Cosmetics, Science and Technology, 2nd Edition, Vol. 1, pp. 32-43 (1972), and U.S. Pat. No. 4,919,934, to Deckner et al., issued Apr. 24, 1990, both of which are incorporated herein by reference in their entirety. These materials are available from Ruger Chemical Co, (Irvington, N.J.).

When the topical formulations of the present invention contain at least one emollient, each emollient is present in an amount from about 0.1 to 15%, preferably 0.1 to about 3.0, more preferably 0.5, 1.0, or 2.5 wt %. Preferably the emollient is a mixture of cetyl alcohol, isopropyl myristate and stearyl alcohol in a 1/5/2 ratio. The emollient may also be a mixture of cetyl alcohol and stearyl alcohol in a 1/2 ratio.

The topical pharmaceutical compositions may also comprise suitable antioxidants, substances known to inhibit oxidation. Antioxidants suitable for use in accordance with the present invention include, but are not limited to, butylated hydroxytoluene, ascorbic acid, sodium ascorbate, calcium ascorbate, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone and tocopherols such as vitamin E, and the like, including pharmaceutically acceptable salts and esters of these compounds. Preferably, the antioxidant is butylated hydroxytoluene, butylated hydroxyanisole, propyl gallate, ascorbic acid, pharmaceutically acceptable salts or esters thereof, or mixtures thereof. Most preferably, the antioxidant is butylated hydroxytoluene. These materials are available from Ruger Chemical Co, (Irvington, N.J.).

When the topical formulations of the present invention contain at least one antioxidant, the total amount of antioxidant present is from about 0.001 to 0.5 wt %, preferably 0.05 to about 0.5 wt %, more preferably 0.1%.

The topical pharmaceutical compositions may also comprise suitable preservatives. Preservatives are compounds added to a pharmaceutical formulation to act as an anti-microbial agent. Among preservatives known in the art as being effective and acceptable in parenteral formulations are benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. See, e.g., Wallhausser, K.-H., Develop. Biol. Standard, 24:9-28 (1974) (S. Krager, Basel). Preferably, the preservative is selected from methylparaben, propylparaben and mixtures thereof. These materials are available from Inolex Chemical Co (Philadelphia, Pa.) or Spectrum Chemicals.

When the topical formulations of the present invention contain at least one preservative, the total amount of preservative present is from about 0.01 to about 0.5 wt %, preferably from about 0.1 to 0.5%, more preferably from about 0.03 to about 0.15. Preferably the preservative is a mixture of methylparaben and proplybarben in a 5/1 ratio. When alcohol is used as a preservative, the amount is usually 15 to 20%.

The topical pharmaceutical compositions may also comprise suitable chelating agents to form complexes with metal cations that do not cross a lipid bilayer. Examples of suitable chelating agents include ethylene diamine tetraacetic acid (EDTA), ethylene glycol-bis(beta-aminoethyl ether)-N,N,N′,N′-tetraacetic acid (EGTA) and 8-Amino-2-[(2-amino-5-methylphenoxy)methyl]-6-methoxyquinoline-N,N,N′,N′-tetraacetic acid, tetrapotassium salt (QUIN-2). Preferably the chelating agents are EDTA and citric acid. These materials are available from Spectrum Chemicals.

When the topical formulations of the present invention contain at least one chelating agent, the total amount of chelating agent present is from about 0.005% to 2.0% by weight, preferably from about 0.05% to about 0.5 wt %, more preferably about 0.1% by weight.

The topical pharmaceutical compositions may also comprise suitable neutralizing agents used to adjust the pH of the formulation to within a pharmaceutically acceptable range. Examples of neutralizing agents include but are not limited to trolamine, tromethamine, sodium hydroxide, hydrochloric acid, citric acid, and acetic acid. Such materials are available from are available from Spectrum Chemicals (Gardena, Calif.).

When the topical formulations of the present invention contain at least one neutralizing agent, the total amount of neutralizing agent present is from about 0.1 wt % to about 10 wt %, preferably 0.1 wt % to about 5.0 wt %, and more preferably about 1.0 wt %. The neutralizing agent is generally added in whatever amount is required to bring the formulation to the desired pH.

The topical pharmaceutical compositions may also comprise suitable viscosity increasing agents. These components are diffusible compounds capable of increasing the viscosity of a polymer-containing solution through the interaction of the agent with the polymer. CARBOPOL ULTREZ 10 may be used as a viscosity-increasing agent. These materials are available from Noveon Chemicals, Cleveland, Ohio.

When the topical formulations of the present invention contain at least one viscosity increasing agent, the total amount of viscosity increasing agent present is from about 0.25% to about 5.0% by weight, preferably from about 0.25% to about 1.0 wt %, and more preferably from about 0.4% to about 0.6% by weight.

The topical pharmaceutical compositions may also comprise one or more suitable solvents. The ability of any solid substance (solute) to dissolve in any liquid substance (solvent) is dependent upon the physical properties of the solute and the solvent. When solutes and solvents have similar physical properties the solubility of the solute in the solvent will be the greatest. This gives rise to the traditional understanding that “like dissolves like.” Solvents can be characterized in one extreme as non-polar, lipophilic oils, while in the other extreme as polar hydrophilic solvents. Oily solvents dissolve other non-polar substances by Van der Wal interactions while water and other hydrophilic solvents dissolve polar substances by ionic, dipole, or hydrogen bonding interactions. All solvents can be listed along a continuum from the least polar, i.e. hydrocarbons such as decane, to the most polar solvent being water. A solute will have its greatest solubility in solvents having equivalent polarity. Thus, for drugs having minimal solubility in water, less polar solvents will provide improved solubility with the solvent having polarity nearly equivalent to the solute providing maximum solubility. Most drugs have intermediate polarity, and thus experience maximum solubility in solvents such as propylene glycol or ethanol, which are significantly less polar than water. If the drug has greater solubility in propylene glycol (for example 8% (w/w)) than in water (for example 0.1% (w/w)), then addition of water to propylene glycol should decrease the maximum amount of drug solubility for the solvent mixture compared with pure propylene glycol. Addition of a poor solvent to an excellent solvent will decrease the maximum solubility for the blend compared with the maximum solubility in the excellent solvent.

When compounds are incorporated into topical formulations the concentration of active ingredient in the formulation may be limited by the solubility of the active ingredient in the chosen solvent and/or carrier. Non-lipophilic drugs typically display very low solubility in pharmaceutically acceptable solvents and/or carriers. For example, the solubility of some borinic acid complexes in water is less than 0.00025% wt/wt. The solubility of the same borinic acid complexes can be less than about 2% wt/wt in either propylene glycol or isopropyl myristate. In one embodiment of the present invention, diethylene glycol monoethyl ether (DGME) is the solvent used to dissolve the compounds of Formula I. The borinic acid complexes useful in the present formulation are believed to have a solubility of from about 10% wt/wt to about 25% wt/wt in DGME. In another embodiment a DGME water cosolvent system is used to dissolve the compounds of Formula I. The solvent capacity of DGME drops when water is added; however, the DGME/water cosolvent system can be designed to maintain the desired concentration of from about 0.1% to about 5% wt/wt active ingredient. Preferably the active ingredient is present from about 0.5% to about 3% wt/wt, and more preferably at about 1% wt/wt, in the as-applied topical formulations. Because DGME is less volatile than water, as the topical formulation evaporates upon application, the active agent becomes more soluble in the cream formulation. This increased solubility reduces the likelihood of reduced bioavailability caused by the drug precipitating on the surface of the skin.

Liquid forms, such as lotions suitable for topical administration or suitable for cosmetic application, may include a suitable aqueous or nonaqueous vehicle with buffers, suspending and dispensing agents, thickeners, penetration enhancers, and the like. Solid forms such as creams or pastes or the like may include, for example, any of the following ingredients, water, oil, alcohol or grease as a substrate with surfactant, polymers such as polyethylene glycol, thickeners, solids and the like. Liquid or solid formulations may include enhanced delivery technologies such as liposomes, microsomes, microsponges and the like.

Additionally, the compounds can be delivered using a sustained-release system, such as semipermeable matrices of solid hydrophobic polymers containing the therapeutic agent. Various sustained-release materials have been established and are well known by those skilled in the art.

Topical treatment regimens according to the practice of this invention comprise applying the composition directly to the skin at the application site, from one to several times daily.

Formulations of the present invention can be used to treat, ameliorate or prevent conditions or symptoms associated with bacterial infections, acne, inflammation and the like.

Additional Active Agents

The following are examples of the cosmetic and pharmaceutical agents that can be added to the topical pharmaceutical formulations of the present invention. The following agents are known compounds and are readily available commercially.

Anti-inflammatory agents include, but are not limited to, bisabolol, mentholatum, dapsone, aloe, hydrocortisone, and the like.

Vitamins include, but are not limited to, Vitamin B, Vitamin E, Vitamin A, Vitamin D, and the like and vitamin derivatives such as tazarotene, calcipotriene, tretinoin, adapalene and the like.

Anti-aging agents include, but are not limited to, niacinamide, retinol and retinoid derivatives, AHA, Ascorbic acid, lipoic acid, coenzyme Q10, beta hydroxy acids, salicylic acid, copper binding peptides, dimethylaminoethyl (DAEA), and the like.

Sunscreens and or sunburn relief agents include, but are not limited to, PABA, jojoba, aloe, padimate-O, methoxycinnamates, proxamine HCLI, lidocaine and the like. Sunless tanning agents include, but are not limited to, dihydroxyacetone (DHA).

Anti-microbial agents include, but are not limited to, clotrimazole, miconazole nitrate, terbinafine HCL, and the like.

Psoriasis-treating agents and/or acne-treating agents include, but are not limited to, salicylic acid, benzoyl peroxide, coal tar, selenium sulfide, zinc oxide, pyrithione (zinc and/or sodium), tazarotene, calcipotriene, tretinoin, adapalene and the like.

Agents that are effective to control or modify keratinization, including without limitation: tretinoin, tazarotene, and adapalene.

The compositions comprising an active agent of Formula I, and optionally at least one of these additional agents, are to be administered topically. In a primary application, this leads to the boronic acid and any other active agent working upon and treating the skin. Alternatively, any one of the topically applied active agents may also be delivered systemically by transdermal routes.

In such compositions an additional cosmetically or pharmaceutically effective agent, such as an anti-inflammatory agent, vitamin, anti-aging agent, sunscreen, anti-microbial agent, and/or acne-treating agent, for example, is usually a minor component (from about 0.001% to about 20% by weight or preferably from about 0.01% to about 10% by weight) with the remainder being various vehicles or carriers and processing aids helpful for forming the desired dosing form.

General Synthetic Methods

The methods of this invention employ readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art using routine optimization procedures.

Additionally, the methods of this invention may employ protecting groups as necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, if the compounds of this invention contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989). Specifically, the compounds of this invention may be prepared by various methods known in the art of organic chemistry in general and nucleoside and nucleotide analogue synthesis in particular. General reviews of the preparation of nucleoside and nucleotide analogues include 1) Michelson A. M. The Chemistry of Nucleosides and Nucleotides, Academic Press, New York, 1963; 2) Goodman L. Basic Principles in Nucleic Acid Chemistry, Academic Press, New York, 1974, Vol. 1, Ch. 2; and 3) Synthetic Procedures in Nucleic Acid Chemistry, Eds. Zorbach W. & Tipson R., Wiley, New York, 1973, Vol. 1 & 2.

As shown in Scheme 1 below, the borinic acid complexes can be obtained form the precursor borinic acids by reaction with one equivalent of the desired heterocyclic ligand in suitable solvents (i.e., ethanol, isopropanol, dioxane, ether, toluene, dimethylformamide, N-methylpyrrolidone, or tetrahydrofuran.

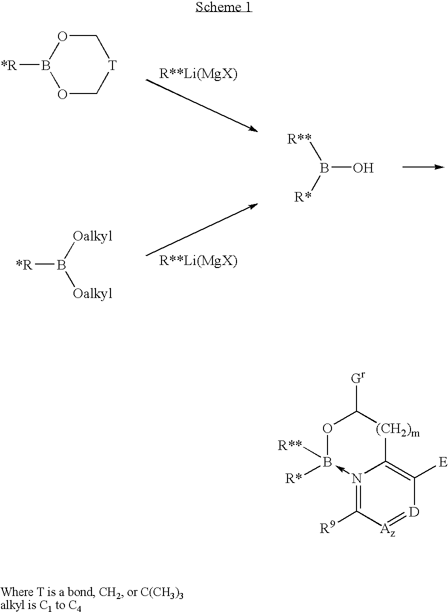

In certain situations, the compounds of the invention may contain one or more asymmetric carbon atoms, so that the compounds can exist in the different stereoisomeric forms. These compounds can be, for example, racemates or optically active forms. In these situations, the single enantiomers, i.e. optically active forms, can be obtained by asymmetric synthesis or by resolution of the racemates. Resolution of the racemates can be accomplished, for example, by conventional methods such as crystallization in the presence of a resolving agent, or chromatography, using for example a chiral HPLC column.

### Utility, Testing, and Administration

Utility

The compounds of the present invention have been implicated in the inhibition of key microbial enzymes, such as bacterial DNA methyltransferase. Many of the compounds disclosed herein are selective inhibitors of methyltransferases in microbes, while not inhibitory for methyltransferases in mammals. However, the anti-bacterial and anti-fungal activity of the compounds of the invention is not limited to those with said enzyme inhibitory activity, nor is the latter effect necessarily essential to said therapeutic activity.

In vitro assays show the active compounds to be active against P. acnes and against other microorganisms commonly found colonizing the skin that may be involved in the pathology of acne. The topical formulations of the present invention represent a novel treatment for acne and/or secondarily infected skin conditions.

Testing

Preferred compounds for use in the present topical formulations will have certain pharmacological properties. Such properties include, but are not limited to, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcová et al. (1996, J. Chromat. B677: 1-27). Compound half-life is inversely proportional to the frequency of dosage of a compound. In vitro half-lives of compounds may be predicted from assays of microsomal half-life as described by Kuhnz and Gleschen (Drug Metabolism and Disposition, (1998) Volume 26, pages 1120-1127).

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective In 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch. 1, p. 1).

Administration

For any compound used in the method of the invention, the therapeutically effective dose can be estimated initially from cell culture assays, as disclosed herein. For example, a dose can be formulated in animal models to achieve a circulating concentration range that includes the EC₅₀ (effective dose for 50% increase) as determined in cell culture, i.e., the concentration of the test compound which achieves a half-maximal inhibition of bacterial cell growth. Such information can be used to more accurately determine useful doses in humans.

In general, the compounds prepared by the methods, and from the intermediates, described herein will be administered in a therapeutically or cosmetically effective amount by any of the accepted modes of administration for agents that serve similar utilities. It will be understood, however, that the specific dose level for any particular patient will depend upon a variety of factors including the activity of the specific compound employed, the age, body weight, general health, sex, diet, time of administration, route of administration, and rate of excretion, drug combination, the severity of the particular disease undergoing therapy and the judgment of the prescribing physician. The drug can be administered from once or twice a day, or up to 3 or 4 times a day.

Dosage amount and interval can be adjusted individually to provide plasma levels of the active moiety that are sufficient to maintain bacterial cell growth inhibitory effects. Usual patient dosages for systemic administration range from 0.1 to 1000 mg/day, preferably, 1-500 mg/day, more preferably 10-200 mg/day, even more preferably 100-200 mg/day. Stated in terms of patient body surface areas, usual dosages range from 50-91 mg/m²/day.

The amount of the compound in a formulation can vary within the full range employed by those skilled in the art. Typically, the formulation will contain, on a weight percent (wt %) basis, from about 0.01-10 wt % of the drug based on the total formulation, with the balance being one or more suitable pharmaceutical excipients. Preferably, the compound is present at a level of about 0.1-3.0 wt %, more preferably, about 1.0 wt %.

### EXAMPLES

While the present invention has been described with respect to preferred embodiments thereof, it will be understood that various changes and modifications will be apparent to those skilled in the art, and that it is intended that the invention encompass such changes and modifications as failing within the scope of the appended claims. The following non-limiting examples are provided to further illustrate the present invention.

The examples below as well as throughout the application, the following abbreviations have the following meanings. If not defined, the terms have their generally accepted meanings.

EDTA Ethylenediamine tetraacetic acid

L=Liter(s)

kg=Kilogram(s)

N=Normal

h=hour

min=Minute

steareth Polyoxyethylene stearyl ether

wt=Weight

wt/wt=Weight to weight ratio

v:v=Volume to volume ratio

In addition, all reaction temperatures and melting points are in degrees Celsius unless reported otherwise and all percentages are molar percents again unless indicated otherwise.

The individual compounds and/or components of the formulations, other than the active ingredient, described herein may be purchased from commercial vendors including, but not limited to Ruger Chemical Co., Inc., Irvington, N.J.; Inolex Chemical Company, Philadelphia, Pa.; Spectrum Chemicals, Gardena, Calif.; Gattefossé Corporation, Westwood, N.J.; Noveon, Inc, Cleveland, Ohio.

### Example 1

### Preparation of 3-hydroxypyridine-2-carbonyloxy-bis(3-chloro-4-methylphenyl)-borane (Compound 1)

Preparation of the Grignard reagent
1. Charge Mg (3.7 equivalent) and tetrahydrofuran (36 L/kg Mg) to a suitable reactor at ambient temperature.
2. Charge a solution of 4-bromo-2-chlorotoluene (3.5 equivalent) in tetrahydrofuran (1.9 L tetrahydrofuran/kg 4-bromo-2-chlorotoluene). The mixture exotherms; adjust addition to control reflux. Cessation of reflux occurs shortly after the addition of the 4-bromo-2-chlorotoluene is complete indicating the Grignard reagent formation is complete. A small amount of Mg metal remains in an otherwise pale, clear Grignard reagent solution.

Preparation of 3-chloro-4-methylphenylborinic acid
3. Cool the Grignard reagent solution below 10° C.
4. Charge a solution of trimethylborate (1.0 equivalent) in tetrahydrofuran (7.7 L tetrahydrofuran/kg trimethylborate).
5. Mix at 40-50° C. for 16-20 h.
6. Cool below 10° C.
7. Charge methanol (12 equivalents).
8. Distill the tetrahydrofuran and methanol under vacuum.
9. Partition the resulting syrup between methyl tert-butyl ether (27 L/kg trimethyl borate) and 1 N HCl (27 L/kg trimethylborate).
10. Adjust the aqueous layer to pH ≦1 with concentrated HCl.
11. Separate the layers and send the aqueous layer to waste.
12. Distill the methyl tert-butyl ether layer under vacuum.
13. Charge toluene (17 L toluene/kg trimethylborate) and distill under vacuum to assist removal of tetrahydrofuran, methanol, methyl tert-butyl ether and water.
14. Dissolve the resultant syrup in ethanol (8 L/kg of active agent).
15. Charge activated carbon (5 wt % based upon 3-hydroxypicolinic acid) (see below). Reflux the mixture for approximately 5-10 min and then filter to remove activated carbon.
16. Charge 3-hydroxypicolinic acid (1.0 equivalent), water (4 L/kg active agent) and ethanol (4 L/kg active agent). Heat to 40-50° C. for approximately 15 min to effect solution.
17. Charge activated carbon (5 wt % based upon 3-hydroxypicolinic acid) and stir approximately 15 min post-activated carbon addition. Filter to remove the activated carbon
18. Charge the filtered, carbon-treated, 3-hydroxypicolinic acid solution to a suitable glass reactor.
19. Charge the filtered, carbon treated, 3-chloro-4-methylphenylborinic acid solution from step 15.
20. Heat the mixture. At approximately 35-45° C. a precipitate forms which then dissolves as the mixture is heated to reflux (approximately 81° C.). Upon reaching reflux an effectively clear solution is obtained. The mixture is refluxed for approximately 15 min.
21. The solution is allowed to cool. At approximately 70-75° C., the solution is seeded with Compound 1. Crystallization occurs as the mixture cools to approximately 25° C. over approximately 10-15 h. The crystalline slurry is held at ambient for approximately 12-15 h. The product slurry is filtered and washed with cold (approximately 5° C.) ethanol/water (3:1 v:v) (1-2 L/kg active agent).
22. The wet cake is dried in trays at ambient temperature without applied vacuum. Heating and vacuum are optional, but not required, to effect drying of the solid.
23. The material is blended and packaged in tight, light resistant containers at normal room temperature.

### Formulation Examples

### Example A

A cream formulation was compounded so that the final product contained the following excipients on the basis of a weight per weight percentage. An aqueous solution containing methylparaben (0.15%), propylparaben (0.03%) and EDTA sodium (0.1%) with dispersed carbomer (CARBOPOL ULTREZ 10, 0.4%) was heated to about 70° C. In a separate container cetyl alcohol (0.5%), isopropyl myristate (2.5%), stearyl alcohol (1.0%) butylated hydroxytoluene (0.1%), BRIJ 721 (1.8%) and BRIJ 72 (1.0%) was heated to about 70° C. to form a clear oil solution. The oil solution was added to the aqueous solution with homogenization for a minimum of five minutes at about 70° C. Cooling of the batch was initiated and with continued mixing the appropriate amount of a 25% trolamine solution (1.0%) was added. To the above mixture as it slowly cooled was added diethylene glycol monoethyl ether (15%). Mixing was continued until a smooth and homogeneous room temperature cream was formed that contained about 75.42% water.

### Example B

A cream formulation was compounded so that the final product contained the following excipients on the basis of a weight per weight percentage. An aqueous solution containing methylparaben (0.15%), propylparaben (0.03%) and EDTA sodium (0.1%) with dispersed carbomer (CARBOPOL ULTREZ 10, 0.4%) was heated to about 70° C. In a separate container cetyl alcohol (0.5%), isopropyl myristate (2.5%), stearyl alcohol (1.0%) butylated hydroxytoluene (0.1%), BRIJ 721 (1.8%) and BRIJ 72 (1.0%) was heated to about 70° C. to form a clear oil solution. The oil solution was added to the aqueous solution with homogenization for a minimum of five minutes at about 70° C. Cooling of the batch was initiated and with continued mixing the appropriate amount of a 25% trolamine solution (1.0%) was added. In a separate vessel compound 1 (1.0%) was completely dissolved in diethylene glycol monoethyl ether (15%) and quantitatively added to the cooling emulsion. Mixing was continued until a smooth and homogeneous room temperature cream was formed that contained about 75.42% water.

### Example C

A cream formulation was compounded so that the final product contained the following excipients on the basis of a weight per weight percentage. The aqueous solution and oil solution of Example A were homogenized for a minimum of five minutes at about 70° C. Cooling of the batch was initiated and with continued mixing the appropriate amount of a 25% trolamine solution (1.0%) was added. In a separate vessel compound 1 (0.1%) was completely dissolved in diethylene glycol monoethyl ether (15%) and quantitatively added to the cooling emulsion. Mixing was continued until a smooth and homogeneous room temperature cream was formed that contained about 76.32% water.

### Example D

A cream formulation was compounded so that the final product contained the following excipients on the basis of a weight per weight percentage. An aqueous solution containing methylparaben (0.15%), propylparaben (0.03%), benzyl alcohol (1.5%), and EDTA sodium (0.1%) with dispersed carbomer (CARBOPOL ULTREZ 10, 0.4%) and acrylates/C10-30 alkyl acrylates crosspolymer (0.2%) was heated to about 70° C. In a separate container cetyl alcohol (0.5%), stearyl alcohol (1.0%) butylated hydroxytoluene (0.1%) was heated to about 70° C. to form a clear oil solution. The oil solution was added to the aqueous solution with homogenization for a minimum of five minutes at about 70° C. Cooling of the batch was initiated and with continued mixing the appropriate amount of a 25% trolamine solution (1.0%) was added. In a separate vessel compound 1 (1.0%) was completely dissolved in diethylene glycol monoethyl ether (9%) and quantitatively added to the cooling emulsion. Mixing was continued until a smooth and homogeneous room temperature cream was formed that contained about 85.02% water.

### Example E

A cream formulation was compounded so that the final product contained the following excipients on the basis of a weight per weight percentage. An aqueous solution containing methylparaben (0.15%), propylparaben (0.03%), benzyl alcohol (2%), and EDTA sodium (0.1%) with dispersed carbomer (CARBOPOL ULTREZ 10, 0.4%) and acrylates/C10-30 alkyl acrylates crosspolymer (0.1%) was heated to about 70° C. In a separate container cetyl alcohol (0.25%), stearyl alcohol (0.5%) butylated hydroxytoluene (0.1%) and polyoxyethylene-4 lauryl ether (3%) was heated to about 70° C. to form a clear oil solution. The oil solution was added to the aqueous solution with homogenization for a minimum of five minutes at about 70° C. Cooling of the batch was initiated and with continued mixing the appropriate amount of a 25% trolamine solution (1.0%) was added. In a separate vessel compound 1 (1.0%) was completely dissolved in diethylene glycol monoethyl ether (8%) and quantitatively added to the cooling emulsion. Mixing was continued until a smooth and homogeneous room temperature cream was formed that contained about 83.37% water.

### Example F

A cream formulation was compounded so that the final product contained the following excipients on the basis of a weight per weight percentage. An aqueous solution containing methylparaben (0.15%), propylparaben (0.03%), benzyl alcohol (2%), and EDTA sodium (0.1%) with dispersed carbomer (CARBOPOL ULTREZ 10, 0.4%) and acrylates/C10-30 alkyl acrylates crosspolymer (0.1%) was heated to about 70° C. In a separate container cetyl alcohol (0.5%), stearyl alcohol (1%), butylated hydroxytoluene (0.1%), BRIJ 721 (1.8%), and BRIJ 72 (1.0%) and polyoxyethylene-4 lauryl ether (2%) was heated to about 70° C. to form a clear oil solution. The oil solution was added to the aqueous solution with homogenization for a minimum of five minutes at about 70° C. Cooling of the batch was initiated and with continued mixing the appropriate amount of a 25% trolamine solution (1.0%) was added. In a separate vessel compound 1 (1.0%) was completely dissolved in diethylene glycol monoethyl ether (8%) and quantitatively added to the cooling emulsion. Mixing was continued until a smooth and homogeneous room temperature cream was formed that contained about 80.82% water.

### Example G

A cream formulation was compounded so that the final product contained the following excipients on the basis of a weight per weight percentage. An aqueous solution containing methylparaben (0.15%), propylparaben (0.03%) and EDTA sodium (0.1%) with dispersed carbomer (CARBOPOL ULTREZ 10, 0.6%) and acrylates/C10-30 alkyl acrylates crosspolymer (0.2%), was heated to about 70° C. In a separate container cetyl alcohol (0.5%), light mineral oil (3%), stearyl alcohol (1.0%) and butylated hydroxytoluene (0.1%) was heated to about 70° C. to form a clear oil solution. The oil solution was added to the aqueous solution with homogenization for a minimum of five minutes at about 70° C. Cooling of the batch was initiated and with continued mixing the appropriate amount of a 25% trolamine solution (1.5%) was added. In a separate vessel compound 1 (1.0%) was completely dissolved in diethylene glycol monoethyl ether (15%) and quantitatively added to the cooling emulsion. Mixing was continued until a smooth and homogeneous room temperature cream was formed that contained about 76.82% water.

### Example H

A topical solution was made by dissolving 1% of compound 1, 0.1% of butylated hydroxytoluene, 0.1% of EDTA disodium, 3% of polysorbate 80, and 3% of polyoxyethylene-4 lauryl ether into 70% ethyl alcohol and about 22.8% water. The pH of the solution was adjusted to pH 5.5.

### Example I

A topical solution was made by dissolving 1% of compound 1, 0.1% of butylated hydroxytoluene, 0.1% of EDTA disodium, and 3% of polyoxyethylene-4 lauryl ether into 70% ethyl alcohol and about 25.8% water. The pH of the solution was adjusted to pH 5.5.

### Example J

A gel formulation was made by combining compound 1 (1%), methylparaben (0.15%), propylparaben (0.03%), benzyl alcohol (2%), polysorbate 80 (2%), and glycerin (5%) with mixing until a clear solution containing no undissolved solids was formed. To the above solution carbomer (CARBOPOL ULTREZ 10, 0.6%) was dispersed and the pH adjusted to 5.5 with a 10% sodium hydroxide solution.

What is claimed is:

1. A topical pharmaceutical formulation comprising a pharmaceutically acceptable topical carrier and a compound having the formula: 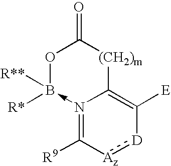
and its pharmaceutically acceptable salts,
wherein:
R* and R** are members independently selected from substituted or unsubstituted aralkyl, substituted or unsubstituted aryl, substituted or unsubstituted cycloalkyl, and substituted or unsubstituted heterocycle;
z is 0 or 1, with the proviso, that
if z is 1, then A is a member selected from CR¹⁰ and N, and D is a member selected from N and CR¹²; and with the further proviso, that
if z is 0, then D is a member selected from O, S, and NR^{12a}.
E is a member selected from hydrogen, hydroxy, alkoxy, (cycloalkyl)oxy, (cycloheteroalkyl)oxy, carboxy, or alkyloxycarbonyl;
m is 0 or 1;
R¹² is a member selected from hydrogen, hydroxyalkyl, aminoalkyl, alkylaminoalkyl, dialkylaminoalkyl, carboxy, alkyloxycarbonyl, amido, hydroxy, alkoxy, aryloxy, thio, alkylthio, arylthio, alkylsulfonyl, dialkylaminosulfonyl, alkylaminosulfonyl, aminosulfonyl, sulfo, cyano, halo, nitro, amino, dialkylamino, alkylamino, arylamino, diarylamino, aralkylamino, and diaralkylamino, wherein the alkyl or aryl portion of any moiety recited for R¹² is optionally substituted;
R^{12a} is a member selected from hydrogen, substituted or unsubstituted alkyl, substituted or unsubstituted heteroalkyl, substituted or unsubstituted aralkyl, substituted or unsubstituted aryl, substituted or unsubstituted cycloalkyl, and substituted or unsubstituted heterocycle;
R⁹ and R¹⁰ are members independently selected from hydrogen, alkyl, cycloalkyl, hydroxyalkyl, aminoalkyl, alkylaminoalkyl, dialkylaminoalkyl, halo, carbonyl, hydroxyimino, carboxy, alkyloxycarbonyl, alkylthio, alkylsulfonyl, arylthio, dialkylaminosulfonyl, alkylaminosulfonyl, aminosulfonyl, amino, alkoxy, nitro, sulfo, and hydroxy, wherein the alkyl or aryl portion of any moiety recited for R⁹ or R¹⁰ is optionally substituted.

2. The topical pharmaceutical formulation of claim 1, wherein the compound has a structure according to the following formula: 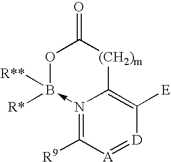
wherein
D is a member selected from N and CR¹².

3. The topical pharmaceutical formulation of claim 1, wherein the compound has a structure according to the following formula: 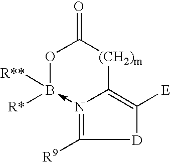
wherein
D is a member selected from O, S and NR^{12a}.

4. The topical pharmaceutical formulation of claim 1 wherein the pharmaceutically acceptable topical carrier comprises at least one solvent in which the compound is soluble.

5. The topical pharmaceutical formulation of claim 1 wherein the pharmaceutically acceptable topical carrier comprises at least one solvent in which the compound has a solubility of at least about 10% wt/wt.

6. The topical pharmaceutical formulation of claim 5 wherein said solvent is miscible with water.

7. The topical pharmaceutical formulation of claim 6 wherein said solvent is diethylene glycol monoethyl ether.

8. The topical pharmaceutical formulation of claim 7 further comprising water in an amount that allows the amount of the compound dissolved in the topical pharmaceutical formulation to be equivalent to at least about 1% wt/wt.

9. The topical pharmaceutical formulation of claim 1, wherein the pharmaceutically acceptable topical carrier comprises: an emollient, an antioxidant, an emulsifier, a preservative, a chelating agent, a viscosity increasing agent, and a neutralizing agent.

10. The topical pharmaceutical formulation of claim 9, wherein the pharmaceutically acceptable topical carrier comprises: cetyl alcohol, isopropyl myristate, stearyl alcohol, butylated hydroxytoluene, polyoxyethylene (2) stearyl ether (Brij 72), polyoxyethylene (21) stearyl ether (Brij 721), methylparaben, propylparaben, EDTA, diethylene glycol monoethyl ether, CARBOPOL ULTREZ 10, 25% trolamine solution, and water.

11. The topical pharmaceutical formulation of claim 2 wherein R⁹ is hydrogen, A is CH, D is CH, E is hydroxy, and m is 0.

12. The topical pharmaceutical formulation of claim 11 wherein R* and R** are the same.

13. The topical pharmaceutical formulation of claim 12 wherein R* and R** are substituted or unsubstituted aryl.

14. The topical pharmaceutical formulation of claim 13 wherein R* and R** are substituted or unsubstituted phenyl, wherein said substituted or unsubstituted phenyl has the structure 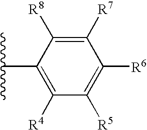
wherein
each of R⁴-R⁸ is a member independently selected from hydrogen, alkyl, cycloalkyl, aryl, substituted aryl, aralkyl, substituted aralkyl, hydroxyalkyl, aminoalkyl, alkylaminoalkyl, dialkylaminoalkyl, carboxy, alkylcarbonyl, aminocarbonyl, alkylaminocarbonyl, dialkylaminocarbonyl, hydroxy, alkoxy, aryloxy, thio, alkylthio, arylthio, alkylsulfonyl, diaminosulfonyl, alkylaminosulfonyl, aminosulfonyl, sulfo, cyano, halo, nitro, amino, 2°-amino, 3°-amino, aminosulfonyl, aminoalkyloxy, (alkylamino)alkyloxy, (dialkylamino)alkyloxy, and cycloheteroalkyl, wherein the alkyl or aryl portion of each moiety recited for R⁴-R⁸ is optionally substituted.

15. The topical pharmaceutical formulation of claim 14 wherein R* and R** are each 3-chloro-4-methylphenyl.

16. The topical pharmaceutical formulation of claim 15, wherein the compound is 3-hydroxypyridine-2-carbonyloxy-bis(3-chloro-4-methylphenyl)-borane.

17. The topical pharmaceutical formulation of claim 14 wherein R* and R** are each 2-methyl-4-chlorophenyl.

18. The topical pharmaceutical formulation of claim 17, wherein the compound is 3-hydroxypyridine-2-carbonyloxy-bis(2-methyl-4-chlorophenyl)-borane.

19. The topical pharmaceutical formulation of claim 1 further comprising a keratinization-modifying agent.

20. A method for treating a patient with a dermatologic condition which method comprises topically administering to said patient a therapeutically effective amount of the topical pharmaceutical formulation of claim 1.

21. The method of claim 20 wherein the condition is an inflammatory condition.

22. The method of claim 20 wherein the condition is acne.

23. The method of claim 20 wherein the condition is secondarily infected skin condition.

24. A topical pharmaceutical formulation, wherein the formulation comprises: an emollient, antioxidant, emulsifiers, preservatives, chelating agent, diethylene glycol monoethyl ether, viscosity increasing agent, neutralizing agent, and water.

25. A topical pharmaceutical formulation, wherein said emollient is a mixture of cetyl alcohol, isopropyl myristate and stearyl alcohol, the antioxidant is butylated hydroxytoluene, the chelating agent is EDTA, the preservatives are a mixture of methylparaben and propylparaben, the emulsifiers are a combination of polyoxyethylene (2) stearyl ether (Brij 72) and polyoxyethylene (21) stearyl ether (Brij 721), said viscosity increasing agent is CARBOPOL ULTREZ 10, said neutralizing agent is trolamine.

26. A method for treating a patient with a dermatologic condition, which method comprises topically administering to said patient a therapeutically effective amount of the topical pharmaceutical formulation of claim 24.

27. The method of claim 26, wherein said condition is pruritis.
